(19) United States
(12) Patent Application Publication
(10) Pub. No.: US 2015/0342743 A1
(43) Pub. Date: Dec. 3, 2015

(54) SHOULDER ENDOPROSTHESIS

(71) Applicant: Kareem Sobky, Denver, CO (US)

(72) Inventor: Kareem Sobky, Denver, CO (US)

(21) Appl. No.: 14/724,301

(22) Filed: May 28, 2015

Related U.S. Application Data
(60) Provisional application No. 62/003,627, filed on May 28, 2014.

(51) Int. Cl. A61F 2/40; A61F 2/30

(52) U.S. Cl. CPC A61F 2/4059; A61F 2/30771; A61F 2002/4062; A61F 2002/4077; A61F 2002/4044; A61F 2002/4037; A61F 2002/404; A61F 2002/30884; A61F 2220/0025; A61F 2002/30906; A61F 2002/3092

(57) ABSTRACT

A shoulder endoprosthesis is disclosed herein. The endoprosthesis is configured as a modular unit of a stem and a cone body. Various sized and shaped stems may be selectively engaged with various sized and shaped cone bodies. Each stem is configured to be accommodated in an intramedullary canal of the humerous. Each cone body is configured to be accommodated in humerous. Each cone body further comprises a channel therethrough, a retaining section disposed within the channel, and an opening in a top end of the cone body configured to receive a mount member from one of a ball head and a reverse baseplate.

### TECHNICAL FIELD

The present disclosure relates generally to a shoulder endoprosthesis device, including an embodiment capable of being used in either an anatomic or reverse application.

### BACKGROUND

Shoulder prostheses are used to restore functionality of an arm. Generally, there are two types of known should prostheses known, anatomical implants and reverse implants. Known anatomical shoulder implants include a stem member having a distal end piece that is insertable into the humerus of a patient and a proximal end piece on which a stem is provided. Traditional anatomical prostheses further include a ball head receivable in the glenoid cavity and this articulates with the ball head that is connected to the stem member. Known reverse implants may also include a stem member with a distal end piece that is insertable into the humerus of a patient. However, reverse implants further include a socket, typically composed of polyethylene, attached via a baseplate. The socket receives a ball head know as a glenoshere that is secured to the glenoid cavity

Traditionally, the stem of the shoulder prosthesis is typically cemented in place in into the humerus. Alternatively, the stem is a single unit (i.e., a monoblock) that is press-fit into the humerus. However, such configurations do not allow for alteration in the version or orientation of the stem. Moreover, there are a limited number of modular humerus components. Even more problematic, is that prior known configurations do not allow for use of the same stem and the same implant body for both anatomic and reverse shoulder implantation. Instead, prior modular systems require explanation of the implant body and revision of a new body to convert to a reverse prosthesis.

However, in many cases there is a need for revision surgery, due to wear of the components, fracture, loosening, infection, improper implantation, rotator cuff tearing in the presence of an anatomic replacement (thereby mandating conversion from an anatomic prosthesis to a reverse prosthesis), etc. With a traditional shoulder prosthesis, when revision is necessary, the cemented or press-fit monoblock stem must be removed and an even longer stem must be re-inserted into its place. However, by cementing a long prosthesis into the humerus, many issues are not addressed such as soft tissue tensioning, version of the stem, proximal bone loss, etc. Long cemented stems may also lead to a very difficult and sometimes disastrous second revision if the original revision fails again. Further, the traditional method of installing shoulder prostheses also does not provide a stable platform to build off of such that would allow for recreation of more normal anatomy. In addition, current modular implants would require removal of the proximal body in the event of an irreparable rotator cuff that requires conversion to a reverse prosthesis. Indeed, once cemented in place, the prosthesis cannot be changed easily without explantation, which can be very traumatic to the patient. What is needed is a prosthesis that addresses the issues above shortcomings, while allowing flexibility for revision surgeries.

### SUMMARY

A shoulder endoprosthesis is disclosed herein. The endoprosthesis is configured as a modular unit of a stem and a cone body. Various sized and shaped stems may be selectively engaged with various sized and shaped cone bodies to allow for the proper fit for individual patients. Each stem is configured to be accommodated in an intramedullary canal of the humerus. Each cone body is configured to be accommodated in the humerus. Each cone body further comprises a channel therethrough, a retaining section disposed within the channel, and an opening in a top end of the cone body configured to receive a mount member from one of a ball head and a reverse baseplate, thereby allowing for more effective conversion surgeries.

### BRIEF DESCRIPTION OF THE DRAWINGS

Embodiments of the present disclosure will now be described by way of example in greater detail with reference to the attached figures, in which:

FIG. 1 is a schematic view of a shoulder endoprosthesis inserted in the humerus;

FIG. 2 is a partially exploded view of the shoulder endoprosthesis of FIG. 1;

FIG. 3 is a schematic view of an exemplary cone body;

FIG. 4 is a front elevational view of an exemplary cone body;

FIG. 5 is a partially exploded view of an anatomic shoulder endoprosthesis assembly inserted in the humerus;

FIG. 6 is a partially exploded view of the anatomic shoulder endoprosthesis application, inserted in the humerus;

FIG. 7 is a schematic view of a reverse shoulder endoprosthesis assembly, utilizing a stem from an anatomic shoulder endoprosthesis; and

FIG. 8 is a schematic view of a reverse shoulder with a shorter cone body than the arrangement of FIG. 7.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

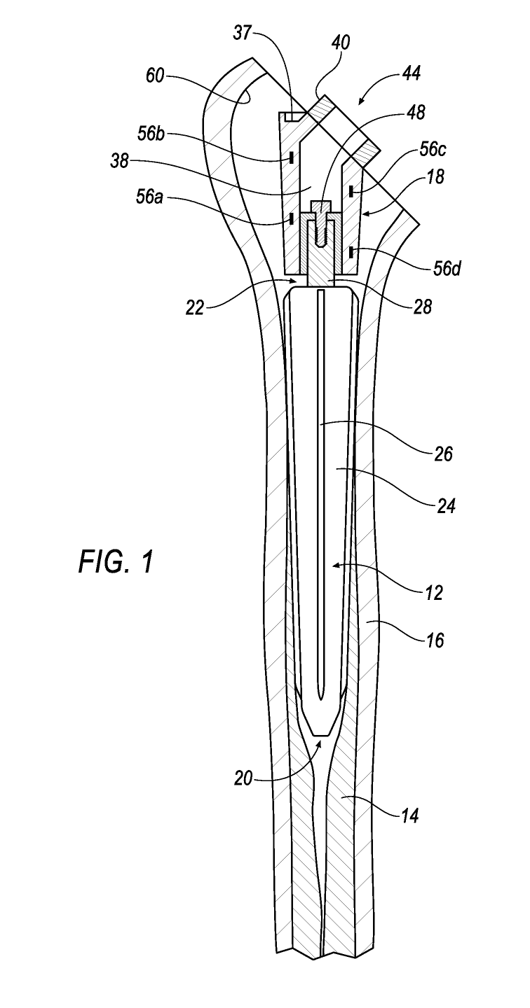

An arrangement for a shoulder endoprosthesis 10 is shown in FIG. 1. The endoprosthesis 10 is configured as a modular assembly in which two independent parts unite to form a solid prosthesis. The entire endoprosthesis 10 is configured to lie within the humeral bone, thereby preserving proximal bone stock. Moreover, the modularity of the endoprosthesis 10 allows for a multitude of sizes to be mixed and matched so as to provide the most stable solution for each individual patient. As a results, endoprosthesis 10 allows the surgeon to treat revision cases or cases of deformity with much more stability of the implant and increased reproducibility of restoring the anatomy of each patient. As will be discussed in further detail below, the endoprosthesis 10 may be used for both anatomic or reverse shoulder applications. The surgeon may need to plan for the possibility of needing to convert to a reverse prosthesis by possibly using a smaller cone body initially, with slightly more bone resection and a thicker ball head, thereby if conversion to a reverse application is needed, over-tensioning the joint can be avoided. The endoprosthesis 10 also allows for treatment of complex fractures of the upper humerus and may also be used for primary shoulder replacements if there is deformity to correct.

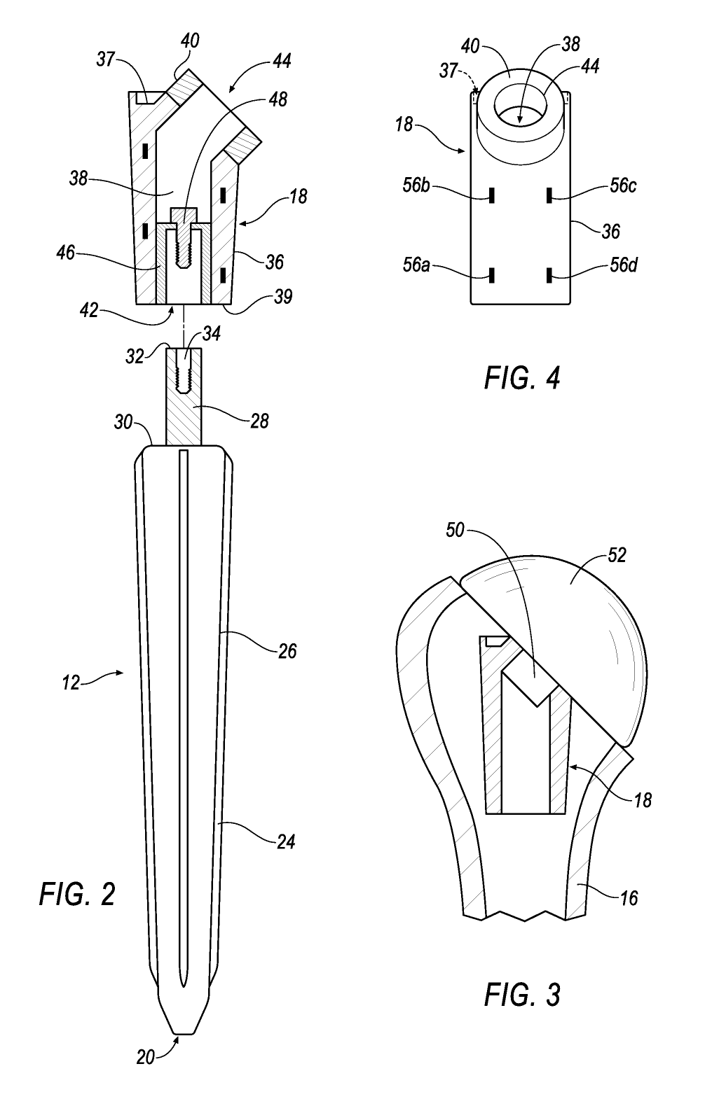

Referring to FIGS. 1 and 2, the endoprosthesis 10 comprises a humeral stem 12 and a cone body 18. The humeral stem 12 is configured to be inserted into an intramedullary canal 14 of the humerus 16. The stem 12 is defined by a distal end 20 and a proximal end 22, with a body section 24 therebetween. In one exemplary configuration, the body section 24 may be provided with one or more splines 26 extending at least a portion of the length of the body section 24. Further, the body section 24 may also be grit blasted, or porous coated (whether splines 26 are included or not). The splines 26 and other coatings for the body section 24 allow for optimal fixation within the humerus 16, as will be discussed in further detail below. The body section 24 may also be tapered from the proximal end 22 to the distal end 20. In one exemplary configuration, the distal end 20 includes an anterior bevel (shown in phantom in FIG. 1) to fit the distal humerus anatomy. It is understood that stem 12 may be configured in a number of different lengths and diameters, making stem 12 particularly useful for revision surgery, though not limited to such.

The proximal end 22 includes an attachment portion 28 that has a diameter that is smaller than a diameter of the proximal end 22 of the stem 12, such that a mounting face 30 is formed. The attachment portion 28 may be configured as a male Morse taper. Extending inwardly from a proximal face 32 of the attachment portion 28 is a connection opening 34. In one exemplary arrangement, the connection opening 34 is threaded.

The cone body 18 is configured to mate with any sized stem 12, thus making the endoprosthesis 10 a truly modular unit. Moreover, it is also contemplated that cone body 18 may be provided in different sizes and diameters. thereby allowing for greater flexibility in providing the best fitting assembly for each individual patient. The cone body 18 is defined by an outer surface 36, a top end 37, an inner channel 38, a bottom end 39 and a mounting portion 40. A first opening 42 is formed in the bottom end 37 and a second opening 44 is formed in top end 37. A retaining section 46 is disposed in the channel 38. Retaining section 46 is configured to receive in an engaging manner the attachment portion 28 of stem 12. In one exemplary arrangement, the retaining section 46 is configured with a female Morse taper. A fastener 48, such as a drop in screw, is configured to extend into the opening 34 and retain the stem to the cone body 18 by engaging with the connection opening 34 of the stem.

The top end 37 has the mounting portion 40 angled with respect to the channel 38. Further, the second opening 44 may be configured as a Morse taper that is configured to accept a male Morse taper of an anatomic ball head or a reverse base plate. For example, the second opening 44 cooperates to receive a mount member 50 of an anatomic ball head 52, as best seen in FIG. 3. Medial front to back suture holes 54a-54d may be formed through the outer surface 36 (as shown in FIG. 4). Medial lateral suture holes 56a-56d may be formed through the outer surface 36 (best seen in FIG. 1).

In the use, the proposed endoprosthesis device 10 provides for the stem 12 to be potted or implanted into the canal 14 of the humerus 16 to gain a stable platform upon which to build on. As discussed above, the stems 12 may be of different diameters and lengths. In one exemplary configuration, the stem 12 and is grit blasted with splines that interdigitate into the cortical bone. This allows will allow for very stable ongrowth/ingrowth of bone. However, it is also understood that the stem 12 may alternatively be cemented. A unique stem feature is that the longer stem choices will be anatomically beveled anteriorly to fit the distal humerus anatomy.

Once the stem 12 is potted, conical reaming of the upper part 60 of the humerus is done with hand or power cone reamers. A cone body 18 of different sizes and diameters may be trialed and implanted for the best fit for the patient. This configuration thus allows for implantation of the cone body 18 within the existing humerus bone 16. A unique feature of the cone body 18 is that version (i.e., the orientation of the cone body 18 within a cylinder) can be placed at any degree and is not dictated by the geometry of the upper part of the humerus 16 or by the body design (such as a broach body or medial/lateral fit type design). This arrangement thereby allows the surgeon to achieve stability and appropriate version and can easily change the version due to the conical shape of the body. Moreover, because the cone body does not rely on medial/lateral fit, it is also bone preserving, which is important for later reverse applications that may need to cut more bone for proper placement. All of the positioning and application of the cone body onto the stem is fully trialed before setting, allowing for selective positioning by the surgeon for the best fit for the individual patient.

When the desired effect and orientation is achieved, then the implantable cone body 18 (which may be plasma porous coated with holes medially and laterally for fracture repair) is implanted. The retaining section 46, which may be configured as an inward Morse tapered junction, has an opening therein, into which a screw or other suitable fastener 48 is dropped in through the top of the cone body 18. The Morse taper of the cone body 18 and the stem 12 is then engaged in the desired version (any version can be set) and the screw 48 is then tightened. Once set, a humeral ball head 52 or reverse baseplate (as discussed below) may engage the inward facing body Morse taper through second opening 44. Notably, the cone body is configured to fit within the humerus 16. Further, unlike prior art designs, if it becomes necessary to perform a later reverse procedure, the configuration of endoprosthesis 10 allows for removal of the humeral ball head 52 and incorporation of a reverse base plate, without necessarily requiring extraction of the cone body.

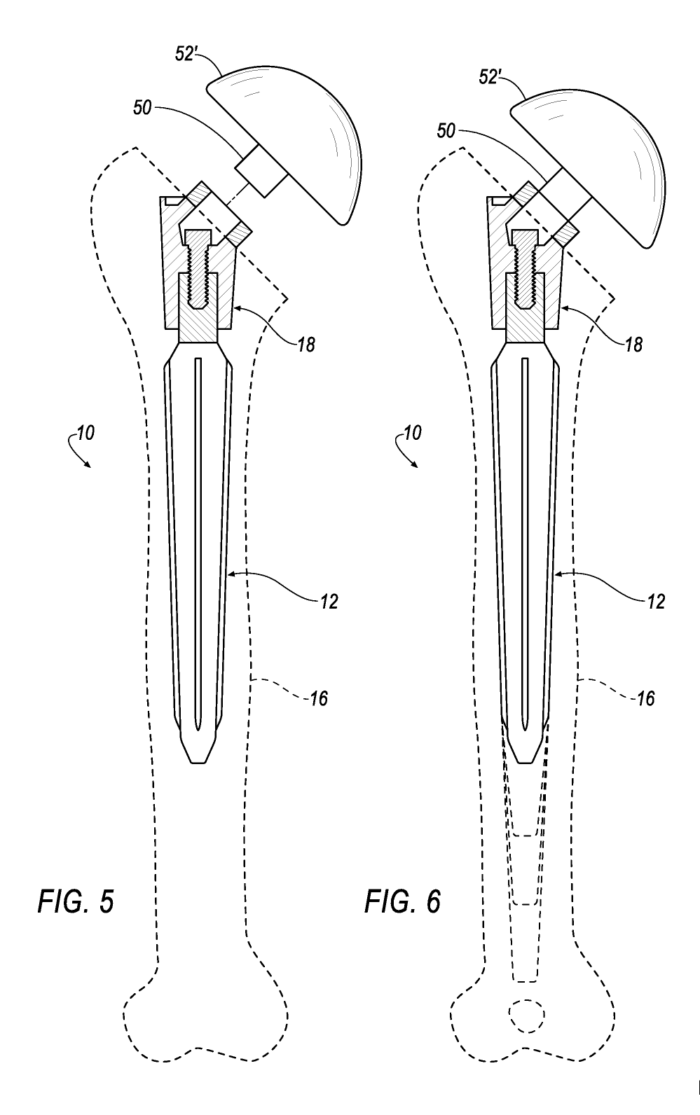

Referring to FIGS. 5-8, application of the endoprosthesis 10 that may be used for both anatomic and reverse applications is illustrated.

As shown in FIGS. 5 and 6, endoprosthesis 10′ comprises a stem 12, a cone body 18 and a humeral head 52′. The humeral head 52′ is thicker than shown in FIG. 4. This configuration allows for the use of a shorter cone body 18. With a shorter cone body 18, slight increased bone resection may be done and it becomes easier to perform a later conversion to a reverse application, as typically with reverse applications there will be a need to cut more bone. The humeral head 52′ is offset in that it includes a male adapter that is not centered in the head 52′. More specifically, the mail adapter is offset from the center to allow the surgeon to dial or manipulate the eccentricity created to match an individual patient's anatomy.

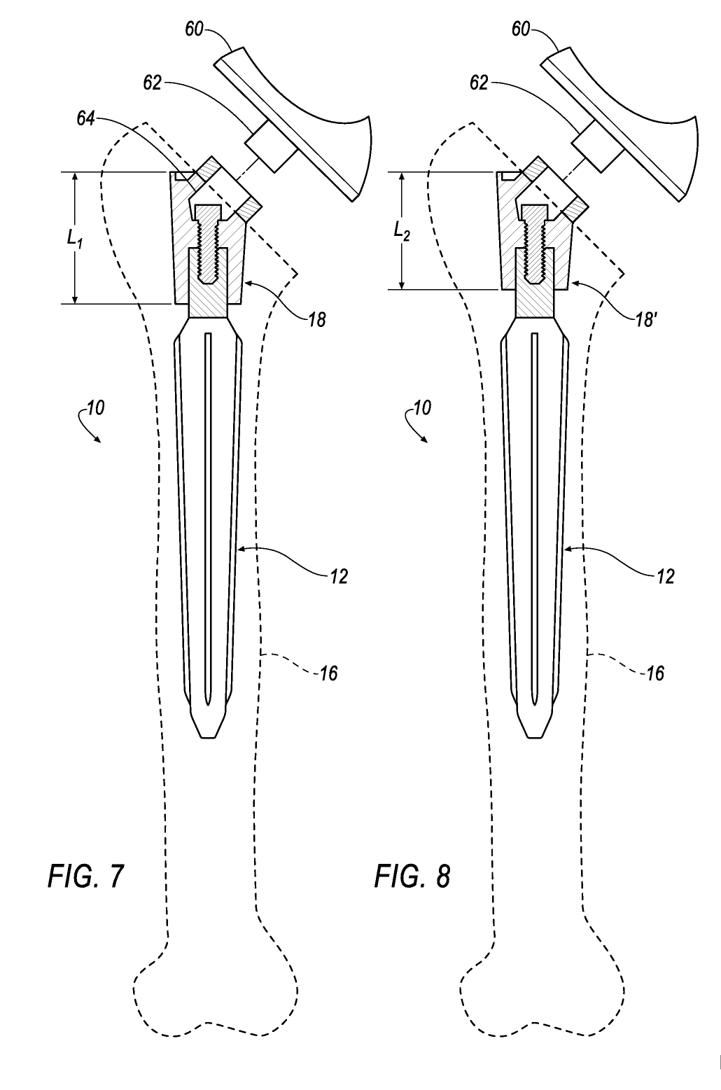

However, if a revision is necessary, endoprosthesis 10 may be easily converted to a reverse application, as shown in FIGS. 7 and 8. For example, if conversion is necessary, the humeral head 52′ is simply removed from the cone body 18. The cone body 18 and the stem 12 may remain in place in the in humerus 16. A base plate 60 having a male adapter 62 is mated with the female adapter 64 positioned in opening 44. The base plate 60, which is typically constructed of metal, such as titanium, supports a socket 66. The socket 66, which is constructed of polyethylene, is secured to the base plate 60 by any known method. The endoprosthesis 10 allows for conversion from a 135° neck for an anatomic application (FIGS. 5-6) to a 143° neck angle for a reverse application.

In some applications, when converting from an anatomic application to a reverse application, it may be desirable to change the cone body 18. More specifically, it may be desirable to use a different length cone body 18. With the modular design of the endoprosthesis, such changes are easily accomplished, without the need for explantation of the stem 12.

The configuration described herein thus provides a modular shoulder prosthetic arrangement in which two independent parts, i.e, the stem 12 and the cone body 18, unite to form a solid prosthesis. Moreover, the entire prosthesis is configured to lie within the humeral bone. The modularity of the arrangement depicted herein allow for a multitude of sizes to be mixed and matched to provide the most stable solution for each individual patient. Accordingly, the arrangement described herein allows the surgeon to treat revision cases or cases of deformity with increased stability of the implant and reproduceablity of restoring the anatomy of each patient. The configuration provides anatomic and reverse shoulder applications and allows for revision of the configuration to a reverse application without requiring explantation of the cone body 18 or the stem 12. The implant 10 also allows for treatment of complex fractures of the upper humerous and may also be used for primary shoulder replacements if there is a deformity to correct.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A shoulder endoprosthesis, comprising:
a stem that is configured to be accommodated in an intramedullary canal of a humerous, and
a cone body configured to be accommodated in humerus, wherein the cone body further comprises a channel therethrough, a retaining section disposed within the channel, and a proximal opening in a top end of the cone body configured to receive a mount member of one of a ball head or a reverse baseplate interchangeably.

2. The shoulder endoprosthesis of claim 1, wherein the stem further includes a plurality of longitudinally extending splines arranged circumferentially.

3. The shoulder endoprosthesis of claim 2, wherein the splines extend the entire length of the stem.

4. The shoulder endoprosthesis of claim 1, wherein a distal end of the stem includes an anterior bevel.

5. The shoulder endoprosthesis of claim 1, wherein the stem is grit blasted or porous coated.

6. The shoulder endoprosthesis of claim 1, further comprising a fastener disposed within the retaining section, wherein the fastener is configured to secure a proximal end of the stem to the retaining section of the cone body.

7. The shoulder endoprosthesis of claim 1, wherein the opening is configured as a female Morse taper and wherein the mount member is configured as a male Morse taper.

8. The shoulder endoprosthesis of claim 1, wherein the stem is defined by a distal end and a proximal end with a body section therebetween, wherein the body section is tapered inwardly from the proximal end to the distal end.

9. The shoulder endoprosthesis of claim 1, wherein the stem further comprises an attachment portion that extends upwardly from a proximal end of the stem, wherein the attachment portion is received within the retaining section.

10. The shoulder endoprosthesis of claim 9, further comprising a fastener element extending through the retaining section and engaging the attachment portion of the stem to secure the cone body and stem together.

11. The shoulder endoprosthesis of claim 1, wherein the mount member of the ball head is offset from a center point of the ball head so as to offset the ball head from the cone body when attached.

12. A shoulder endoprosthesis kit, comprising:
at least one stem that is configured to be accommodated in an intramedullary canal of a humerus; and
at least one cone body configured to be accommodated in humerus, wherein the cone body further comprises a channel therethrough, a retaining section disposed within the channel;
a ball head; and
a reverse baseplate;
wherein the cone body includes a proximal opening in a top end of the cone body configured to receive a mount member of one of the ball head and the reverse baseplate interchangeably.

13. The shoulder endoprosthesis kit of claim 12, further comprising a socket member mounted to the reverse baseplate.

14. The shoulder endoprosthesis of claim 12, wherein the stem further includes a plurality of longitudinally extending splines arranged circumferentially.

15. The shoulder endoprosthesis of claim 14, wherein the stem is grit blasted or porous coated.

16. The shoulder endoprosthesis of claim 14, wherein a distal end of the stem includes an anterior bevel.
